(19) United States
(12) Patent Application Publication
(10) Pub. No.: US 2019/0125721 A1
(43) Pub. Date: May 2, 2019

(54) METHODS AND COMPOSITIONS FOR TREATMENT OF RETT SYNDROME

(71) Applicant: Neurotrope Bioscience, Inc., New York, NY (US)

(72) Inventors: Jeffrey BENISON, Valley Stream, NY (US); Daniel L. ALKON, Bethesda, MD (US)

(21) Appl. No.: 16/097,806

(22) PCT Filed: May 4, 2017
(86) PCT No.: PCT/US2017/031144

§ 371 Date: Oct. 30, 2018

Related U.S. Application Data
(60) Provisional application No. 62/331,913, filed on May 4, 2016.

(51) Int. Cl. A61K 31/365

(52) U.S. Cl. CPC A61K 31/365

(57) ABSTRACT

The present application provides methods for treating human subjects suffering from Rett Syndrome by administering PKC activators, for example, bryostatin 1, other bryostatins and bryologs. The present disclosure provides, according to certain embodiments, methods comprising administering to a subject with Rett syndrome a pharmaceutically effective amount of bryostatin 1.

### RELATED APPLICATION

This application claims the benefit of and priority to U.S. Provisional Application No. 62/331,913, filed May 4, 2016, which is incorporated by reference herein in its entirety for all purposes.

### FIELD OF THE APPLICATION

This application relates to methods for treating human subjects suffering from Rett Syndrome by administering PKC activators, for example, bryostatin 1. In one embodiment, the application relates to a method comprising administering to a subject with Rett syndrome a pharmaceutically effective amount of bryostatin 1.

### BACKGROUND

Rett Syndrome (RTT) is a neurodevelopmental disorder that almost exclusively affects females (1 in 10,000 live births). RTT is classified as an autism spectrum disorder (Diagnostic and Statistical Manual of Mental Disorders, Fourth Edition-Revised (DSM-IV-R)). Approximately 16,000 patients are currently affected by it in the U.S.A. (Rett Syndrome Research Trust data). For a diagnosis of Rett syndrome, the following symptoms are characteristic: impaired development from age 6-18 months; slowing of the rate of head growth starting from between age 3 months and 4 years; severely impaired language; repetitive and stereotypic hand movements; and gait abnormalities, e.g., toe-walking or unsteady stiff-legged walk. There are a number of supportive criteria that may help diagnosis of Rett Syndrome, but are not essential for a diagnosis. These include breathing difficulties, EEG abnormalities, seizures, muscle rigidity and spasticity, scoliosis (curving of the spine), teeth-grinding, small hands and feet in relation to height, growth retardation, decreased body fat and muscle mass, abnormal sleep patterns, irritability or agitation, chewing and/or swallowing difficulties, poor circulation and constipation.

The onset of RTT usually begins between 6-18 months of age with a slowing of development and growth rates. This is followed by a regression phase (typically in children aged 1-4 years of age), pseudo-stationary phase (2-10 years of age) and a subsequent progressive late motor deterioration state. RTT symptoms include sudden deceleration of growth and regression in language and motor skills including purposeful hand movements being replaced by stereotypical movements, autistic features, panic-like attacks, sleep cycle disturbances, tremors, seizures, respiratory dysfunctions (episodic apnea, hyperpnea), apraxia, dystonia, dyskinesia, hypotonia, progressive kyphosis or scoliosis and severe cognitive impairment. Most RTT patients survive into adulthood with severe disabilities and require 24-hour-a-day care.

Between 85% and 95% cases of RTT are reported to be caused by a mutation of the Mecp2 gene (Amir et al. 1999. Nat Genet 23:185-188; Rett Syndrome Research Trust)—a gene encoding methyl-CpG-binding protein 2 (MeCP2). Mecp2 maps to the X-chromosome (location Xq28) and for this reason, mutations to the gene in males are usually lethal. While RTT is a genetic disorder, less than 1% of recorded cases are inherited; almost all mutations of Mecp2 occur de novo, with two thirds caused by mutations at 8 CpG dinucleotides (R106, R133, T158, R168, R255, R270, R294 and R306) located on the third and fourth exons.

MeCP2 is a protein that binds methylated CpG dinucleotides to exert transcriptional silencing of DNA in the CNS. The key effect of a reduction or absence of MeCP2 appears to be an impairment of dendritic spine development and the formation of synapses. MeCP2 expression appears to temporally correlate with brain maturation, explaining why symptoms typically appear around 18 months of age.

The course of Rett syndrome, including the age of onset and the severity of symptoms, varies from child to child. Before the symptoms begin, however, the child generally appears to grow and develop normally, although there are often subtle abnormalities even in early infancy, such as loss of muscle tone (hypotonia), difficulty feeding, and jerkiness in limb movements. Then, gradually, mental and physical symptoms appear. As the syndrome progresses, the child loses purposeful use of her hands and the ability to speak. Other early symptoms may include problems crawling or walking and diminished eye contact. The loss of functional use of the hands is followed by compulsive hand movements such as wringing and washing. The onset of this period of regression is sometimes sudden. The inability to perform motor functions, i.e., apraxia is perhaps the most severely disabling feature of Rett syndrome, interfering with every body movement, including eye gaze and speech.

Children with Rett syndrome often exhibit autistic-like behaviors in the early stages. Other symptoms may include walking on the toes, sleep problems, a wide-based gait, teeth grinding and difficulty chewing, slowed growth, seizures, cognitive disabilities, and breathing difficulties while awake such as hyperventilation, apnea (breath holding), and air swallowing. Other impairments include a presentation of delayed intellectual development most commonly manifest as a shortfall in language skills. Cognitive loss relative to normal parameters for the age is often quite marked in RTT. The presence of epilepsy or abnormal activity in the EEG is also common to Rett Syndrome. Epilepsy arises in patients suffering from RTT in situations of abnormal neuronal connectivity, impaired neuronal connectivity and deranged synaptic function.

Nearly all cases of Rett syndrome are caused by a mutation in the methyl CpG binding protein 2, or Mecp2, gene. The Mecp2 gene contains instructions for the synthesis of a protein called methyl cytosine binding protein 2 (MeCP2), which is needed for brain development and acts as one of the many biochemical switches that can either increase gene expression or tell other genes when to turn off and stop producing their own unique proteins. Because the Mecp2 gene does not function properly in individuals with Rett syndrome, insufficient amounts or structurally abnormal forms of the protein are produced and can cause other genes to be abnormally expressed.

There is no cure for Rett syndrome. Treatment for the disorder is symptomatic and supportive, requiring a multidisciplinary approach. Medication may be needed for breathing irregularities and motor difficulties, and anticonvulsant drugs may be used to control seizures.

As described above, a conserved pathology is observed in Rett Syndrome patients that comprise impaired neurite development and impaired synaptic connectivity, along with a corresponding impairment in social and cognitive functioning as a result. Such synaptic dysfunctions result from genetically altered functions of postsynaptic density proteins. Normal neurite growth and postsynaptic development may be regulated and augmented by growth factors such as brain derived neurotrophic factor (BDNF; Chapleau et al, 2009). Drugs that promote BDNF function are therefore of use in the treatment of progressive developmental disorders such as RTT.

Thus, there is a need for the development of therapies which activate key synaptic growth factors such as brain-derived neurotrophic factor (BDNF), insulin-like growth factor (IGF), or nerve growth factor (NGF), which regulate and augment normal neurite growth and postsynaptic development in patients suffering from Rett syndrome.

### SUMMARY

The application pertains to the use of a pharmaceutically effective amount of bryostatin 1 in the treatment of Rett syndrome.

The application also pertains to the use of a pharmaceutically effective amount of bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, a bryolog, a polyunsaturated fatty acid, or combinations thereof, in the treatment of Rett syndrome.

The application also pertains to the use of a pharmaceutically effective amount of bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, or combinations thereof, in the treatment of Rett syndrome.

The application also pertains to the use of a pharmaceutically effective amount of bryostatin 1 in the treatment of Rett syndrome, wherein the pharmaceutically effective amount of bryostatin 1 is from about 0.0000001 mg/kg to about 250 mg/kg per dose.

The application also pertains to the use of a pharmaceutically effective amount of bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, a bryolog, a polyunsaturated fatty acid, or combinations thereof, in the treatment of Rett syndrome, wherein the pharmaceutically effective amount of the compound or combination of compounds is from about 0.0000001 mg/kg to about 250 mg/kg per dose.

The application also pertains to the use of a pharmaceutically effective amount of bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, or combinations thereof, in the treatment of Rett syndrome, wherein the pharmaceutically effective amount of the compound or combination of compounds is from about 0.0000001 mg/kg to about 250 mg/kg per dose.

The application also pertains to the use of a pharmaceutically effective amount of bryostatin 1 in the treatment of Rett syndrome, wherein the pharmaceutically effective amount of bryostatin 1 is from about 0.00001 mg/kg to about 5.0 mg/kg per dose.

The application also pertains to the use of a pharmaceutically effective amount of bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, a bryolog, a polyunsaturated fatty acid, or combinations thereof, in the treatment of Rett syndrome, wherein the pharmaceutically effective amount of the compound or combination of compounds is from about 0.00001 mg/kg to about 5.0 mg/kg per dose.

The application also pertains to the use of a pharmaceutically effective amount of bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, or combinations thereof, in the treatment of Rett syndrome, wherein the pharmaceutically effective amount of the compound or combination of compounds is from about 0.00001 mg/kg to about 5.0 mg/kg per dose.

The application also pertains to the use of a pharmaceutically effective amount of bryostatin 1 in the treatment of Rett syndrome, wherein the pharmaceutically effective amount is from about 0.00001 mg/kg to about 5.0 mg/kg per day, 0.00005 mg/kg to about 3.0 mg/kg per dose, 0.0001 mg/kg to about 2.0 mg/kg per day, 0.0005 mg/kg to about 1.5 mg/kg per day, 0.001 mg/kg to about 1.0 mg/kg per day, 0.005 mg/kg to about 0.5 mg/kg per day, or 0.01 mg/kg to about 0.2 mg/kg per day, or 0.01 mg/kg to about 0.1 mg/kg per day. In one embodiment, the pharmaceutically effective amount is administered in a single dose. In one embodiment, the pharmaceutically effective amount is administered in multiple dose. In one embodiment, the pharmaceutically effective amount is administered in a single dose and administered intravenously (IV). In one embodiment, the pharmaceutically effective amount is administered in multiple dose and administered intravenously (IV).

The application also pertains to the use of a pharmaceutically effective amount of bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, a bryolog, a polyunsaturated fatty acid, or combinations thereof, in the treatment of Rett syndrome, wherein the pharmaceutically effective amount is from about 0.00001 mg/kg to about 5.0 mg/kg per day, 0.00005 mg/kg to about 3.0 mg/kg per dose, 0.0001 mg/kg to about 2.0 mg/kg per day, 0.0005 mg/kg to about 1.5 mg/kg per day, 0.001 mg/kg to about 1.0 mg/kg per day, 0.005 mg/kg to about 0.5 mg/kg per day, or 0.01 mg/kg to about 0.2 mg/kg per day, or 0.01 mg/kg to about 0.1 mg/kg per day. In one embodiment, the pharmaceutically effective amount is administered in a single dose. In one embodiment, the pharmaceutically effective amount is administered in multiple dose. In one embodiment, the pharmaceutically effective amount is administered in a single dose and administered intravenously. In one embodiment, the pharmaceutically effective amount is administered in multiple dose and administered intravenously.

The application also pertains to the use of a pharmaceutically effective amount of bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, or combinations thereof, in the treatment of Rett syndrome, wherein the pharmaceutically effective amount is from about 0.00001 mg/kg to about 5.0 mg/kg per day, 0.00005 mg/kg to about 3.0 mg/kg per dose, 0.0001 mg/kg to about 2.0 mg/kg per day, 0.0005 mg/kg to about 1.5 mg/kg per day, 0.001 mg/kg to about 1.0 mg/kg per day, 0.005 mg/kg to about 0.5 mg/kg per day, or 0.01 mg/kg to about 0.2 mg/kg per day, or 0.01 mg/kg to about 0.1 mg/kg per day. In one embodiment, the pharmaceutically effective amount is administered in a single dose. In one embodiment, the pharmaceutically effective amount is administered in multiple dose. In one embodiment, the pharmaceutically effective amount is administered in a single dose and administered intravenously. In one embodiment, the pharmaceutically effective amount is administered in multiple dose and administered intravenously.

The application also pertains to the use of a pharmaceutically effective amount of bryostatin 1 in the treatment of Rett syndrome, wherein the pharmaceutically effective amount of bryostatin 1 is provided in a dose from 0.01-25 μg/m² intravenously (IV).

The application also pertains to the use of a pharmaceutically effective amount of bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, a bryolog, a polyunsaturated fatty acid, or combinations thereof, in the treatment of Rett syndrome, wherein the pharmaceutically effective amount of the compound or combination of compounds is provided in a dose from 0.01-25 μg/m² IV.

The application also pertains to the use of a pharmaceutically effective amount of bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, or combinations thereof, in the treatment of Rett syndrome, wherein the pharmaceutically effective amount of the compound or combination of compounds is provided in a dose from 0.01-25 μg/m² IV.

The application also pertains to a method for activating a synaptic growth factor in a patient suffering from Rett syndrome comprising administering a pharmaceutically effective amount of a PKC activator to said patient, wherein the activation results in a corrective and/or normalizing effect on the brain development in said patient suffering from Rett syndrome.

The application also pertains to a method for activating a synaptic growth factor in a patient suffering from Rett syndrome comprising administering a pharmaceutically effective amount of a PKC activator to said patient, wherein the activation results in an increase in the protein levels of synaptic growth factors in said patient.

The application also pertains to a method for activating a synaptic growth factor in a patient suffering from Rett syndrome comprising administering a pharmaceutically effective amount of a PKC activator to said patient, wherein the activation results in the prevention and/or reduction in neuronal death in said patient.

The protein kinase C (PKC) family of enzymes is responsible for a multitude of cellular processes through the enzymes' ability to regulate proteins via signal transduction cascades. The members of this kinase family are structurally and functionally similar and are categorized into conventional (α, β1, βII and γ), novel (δ, ε, η, and θ), and atypical isoforms (ζ and λ). These isoforms have been implicated in a variety of diseases and pathological conditions. (See Mellor and Parker (1998) Biochem. J. 332(2): 281-292; Azzi et al. (1992) Eur. J. Biochem. 208:547-557; Cloud-Heflin et al. (1996) Eur. J. Biochem. 239: 796-804; and Mochly-Rosen et al. Nat. Rev. Drug Discov. 11: 937-957.)

The PKC ε and PKC α isozymes are responsible for increasing the synthesis of synaptic growth factors including BDNF, IGF, and NGF, thereby increasing the levels of these growth factors. Further, the PKC ε and PKC α isozymes are anti-apoptotic, i.e., they prevent and/or reduce neuronal and synaptic death. In one embodiment, PKC ε contributes more than PKC α towards the increase in the synthesis of synaptic growth factors including BDNF, IGF, and NGF. In one embodiment, PKC ε is more efficacious at preventing and/or reducing neuronal and synaptic death than PKC α.

The present disclosure provides methods for treating human subjects suffering from Rett syndrome, by administering PKC activators.

The present disclosure provides, according to certain embodiments, methods comprising administering to a subject with Rett syndrome a pharmaceutically effective amount of a PKC activator.

The present disclosure provides, according to certain embodiments, methods comprising administering to a subject with Rett syndrome a pharmaceutically effective amount of bryostatin 1.

The features and advantages of the present disclosure will be readily apparent to those skilled in the art upon a reading of the description of the embodiments that follows.

### DETAILED DESCRIPTION

The present disclosure relates to the use of a pharmaceutically effective amount of bryostatin 1 in the treatment of Rett syndrome.

The present disclosure also relates to the use of a pharmaceutically effective amount of bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, a bryolog, a polyunsaturated fatty acid, or combinations thereof, in the treatment of Rett syndrome.

The present disclosure also relates to the use of a pharmaceutically effective amount of bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, or combinations thereof, in the treatment of Rett syndrome

The present disclosure also relates to the use of a pharmaceutically effective amount of bryostatin 1 in the treatment of Rett syndrome, wherein the pharmaceutically effective amount of bryostatin 1 is from about 0.0000001 mg/kg to about 250 mg/kg per dose.

The present disclosure also relates to the use of a pharmaceutically effective amount of bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, a bryolog, a polyunsaturated fatty acid, or combinations thereof, in the treatment of Rett syndrome, wherein the pharmaceutically effective amount of the compound or combination of compounds is from about 0.0000001 mg/kg to about 250 mg/kg per dose.

The present disclosure also relates to the use of a pharmaceutically effective amount of bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, or combinations thereof, in the treatment of Rett syndrome, wherein the pharmaceutically effective amount of the compound or combination of compounds is from about 0.0000001 mg/kg to about 250 mg/kg per dose.

The present disclosure also relates to the use of a pharmaceutically effective amount of bryostatin 1 in the treatment of Rett syndrome, wherein the pharmaceutically effective amount of bryostatin 1 is from about 0.00001 mg/kg to about 5.0 mg/kg per dose.

The present disclosure also relates to the use of a pharmaceutically effective amount of bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, a bryolog, a polyunsaturated fatty acid, or combinations thereof, in the treatment of Rett syndrome, wherein the pharmaceutically effective amount of the compound or combination of compounds is from about 0.00001 mg/kg to about 5.0 mg/kg per dose.

The present disclosure also relates to the use of a pharmaceutically effective amount of bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, or combinations thereof, in the treatment of Rett syndrome, wherein the pharmaceutically effective amount of the compound or combination of compounds is from about 0.00001 mg/kg to about 5.0 mg/kg per dose.

The present disclosure also relates to the use of a pharmaceutically effective amount of bryostatin 1 in the treatment of Rett syndrome, wherein the pharmaceutically effective amount of bryostatin 1 is provided in a dose from 0.01-25 μg/m² intravenously (IV).

The present disclosure also relates to the use of a pharmaceutically effective amount of bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, a bryolog, a polyunsaturated fatty acid, or combinations thereof, in the treatment of Rett syndrome, wherein the pharmaceutically effective amount of the compound or combination of compounds is provided in a dose from 0.01-25 μg/m² IV.

The present disclosure also relates to the use of a pharmaceutically effective amount of bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, or combinations thereof, in the treatment of Rett syndrome, wherein the pharmaceutically effective amount of the compound or combination of compounds is provided in a dose from 0.01-25 μg/m² IV.

The present disclosure also relates to a method for activating a synaptic growth factor in a patient suffering from Rett syndrome comprising administering a pharmaceutically effective amount of a PKC activator to said patient, wherein the activation results in a corrective and/or normalizing effect on the brain development in said patient suffering from Rett syndrome.

For example, the synaptic growth factor is brain-derived neurotrophic factor (BDNF), insulin-like growth factor (IGF), and/or nerve growth factor (NGF).

For example, IGF is IGF-1.

For example, the PKC activator is bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, a bryolog, or any combination thereof.

For example, the PKC activator is bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, or any combination thereof

For example, the PKC activator is bryostatin 1.

For example, the PKC activator further comprises one or more of a polyunsaturated fatty acid, a potassium channel activator, for example, diazoxide, a neristatin, for example, neristatin 1, or any other PKC activator described herein. For example, the PKC activator is a polyunsaturated fatty acid.

For example, the PKC activator is a potassium channel activator.

For example, the PKC activator is a neristatin.

For example, the PKC activator is phorbol-12-myristate-13-acetate (PMA), okadaic acid, 1α,25-dihydroxyvitamin D3,12-deoxyphorbol-13-acetate (prostratin), 1,2-dioctanoyl-sn-glycerol (DOG), 1-oleoyl-2-acetyl-sn-glycerol (OAG), (2S,5S)-(E,E)-8-(5-(4-(trifluoromethyl)phenyl)-2,4-pentadienoylamino)benzolactam (α-amyloid precursor protein modulator), cis-9-octadecenoic acid (oleic acid), ingenol 3-angelate, resiniferatoxin, L-α-Phosphatidyl-D-myo-inosito1-4,5-bisphosphate, triammonium salt (PIP2), phorbol-12,13-dibutyrate, 8(S-hydroxy-(5Z, 9E,11Z,14Z)-eicosatetraenoic acid (8(S)-HETE), 12β-[(E,E)-5-Phenyl-2,4-pentadienoyloxy]daphnetoxin (merzerein), clomiphene citrate, sodium oleate, phorbol 12,13-diacetate, phorbol-12,13-didecanoate, 1,2-dipalmitoyl-sn-glycerol, 1-Stearoyl-2-linoleoyl-sn-glycerol, 1-stearoyl-2-linoleoyl-sn-glycerol, phorbol-12,13-dihexanoate, prostratin, a prostratin analog, resiniferonol 9,13,14-ortho-phenylacetate, C-8 ceramide, 1,6-bis(Cyclohexyloximinocarbonylamino)hexane; 1,6-Di(O-(carbamoyl)cyclohexanone oxime) hexane (RHC-80267), (+/−-oleoyl-2-acetylglycerol, 5(S),6(R),15(S)-TriHETE (Lipoxin A4), (−)-Indolactam V, SC-9, SC-10, zoledronic acid monohydrate, 12-deoxyphorbo-13-angelate 20-acetate, 6-(N-decylamino)-4-hydroxymethylindole, 4α-phorbol 12,13-dibutyrate, 1,2-dihexanoyl-sn-glycerol, zoledronic acid disodium salt tetrahydrate, arachidonic acid methyl ester, or arachidonic acid-d8.

For example, the PKC activator activates the PKC ε isozyme and/or the PKC α isozyme.

For example, the PKC activator activates the PKC ε isozyme. For example, the PKC activator is administered orally, intraperitoneally, subcutaneously, intranasally, buccally, transdermally, intramuscularly, intrarectally, intravenously, or by inhalation.

For example, the PKC activator is administered orally.

For example, the PKC activator is administered intravenously.

For example, the corrective and/or normalizing effect results in an abatement of symptoms arising from a muscular issue, a respiratory issue, a developmental issue, a behavioral issue, and/or a cognitive issue.

For example, the corrective and/or normalizing effect results in an abatement of symptoms arising from epilepsy, seizures, constipation, drooling, scoliosis, teeth grinding, and/or tremors.

The present disclosure also results in a method for activating a synaptic growth factor in a patient suffering from Rett syndrome comprising administering a pharmaceutically effective amount of a PKC activator to said patient, wherein the activation results in an increase in the protein levels of synaptic growth factors in said patient.

For example, the increase in the protein levels of synaptic growth factors in said patient results in a corrective and/or normalizing effect on the brain development in said patient suffering from Rett syndrome.

For example, the corrective and/or normalizing effect results in an abatement of symptoms arising from a muscular issue, a respiratory issue, a developmental issue, a behavioral issue, and/or a cognitive issue.

For example, the corrective and/or normalizing effect results in an abatement of symptoms arising from epilepsy, seizures, constipation, drooling, scoliosis, teeth grinding, and/or tremors.

For example, the synaptic growth factor is brain-derived neurotrophic factor (BDNF), insulin-like growth factor (IGF), and/or nerve growth factor (NGF).

For example, IGF is IGF-1.

For example, the PKC activator is bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, a bryolog, or any combination thereof.

For example, the PKC activator is bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, or any combination thereof

For example, the PKC activator is bryostatin 1.

For example, the PKC activator further comprises one or more of a polyunsaturated fatty acid, a potassium channel activator, for example, diazoxide, a neristatin, for example, neristatin 1, or any other PKC activator described herein.

For example, the PKC activator is a polyunsaturated fatty acid.

For example, the PKC activator is a potassium channel activator.

For example, the PKC activator is a neristatin.

For example, the PKC activator is phorbol-12-myristate-13-acetate (PMA), okadaic acid, 1α,25-dihydroxyvitamin D3,12-deoxyphorbol-13-acetate (prostratin), 1,2-dioctanoyl-sn-glycerol (DOG), 1-oleoyl-2-acetyl-sn-glycerol (OAG), (2S,5S)-(E,E)-8-(5-(4-(trifluoromethyl)phenyl)-2,4-pentadienoylamino)benzolactam (α-amyloid precursor protein modulator), cis-9-octadecenoic acid (oleic acid), ingenol 3-angelate, resiniferatoxin, L-α-Phosphatidyl-D-myo-inosito1-4,5-bisphosphate, triammonium salt (PIP2), phorbol-12, 13-dibutyrate, 8(S-hydroxy-(5Z, 9E,11Z,14Z)-eicosatetraenoic acid (8(S)-HETE), 12β-[(E,E)-5-Phenyl-2,4-pentadienoyloxy]daphnetoxin (merzerein), clomiphene citrate, sodium oleate, phorbol 12,13-diacetate, phorbol-12,13-didecanoate, 1,2-dipalmitoyl-sn-glycerol, 1-Stearoyl-2-linoleoyl-sn-glycerol, 1-stearoyl-2-linoleoyl-sn-glycerol, phorbol-12,13-dihexanoate, prostratin, a prostratin analog, resiniferonol 9,13,14-ortho-phenylacetate, C-8 ceramide, 1,6-bis(Cyclohexyloximinocarbonylamino)hexane; 1,6-Di(O-(carbamoyl)cyclohexanone oxime) hexane (RHC-80267), (+/−)-1-oleoyl-2-acetylglycerol, 5(S),6(R),15(S)-TriHETE (Lipoxin A4), (−)-Indolactam V, SC-9, SC-10, zoledronic acid monohydrate, 12-deoxyphorbo-13-angelate 20-acetate, 6-(N-decylamino)-4-hydroxymethylindole, 4α-phorbol 12,13-dibutyrate, 1,2-dihexanoyl-sn-glycerol, zoledronic acid disodium salt tetrahydrate, arachidonic acid methyl ester, or arachidonic acid-d8.

For example, the PKC activator activates the PKC ε isozyme and/or the PKC α isozyme.

For example, PKC activator activates the PKC ε isozyme.

For example, the PKC activator is administered orally, intraperitoneally, subcutaneously, intranasally, buccally, trans-dermally, intramuscularly, intrarectally, intravenously, or by inhalation.

For example, the PKC activator is administered orally.

For example, the PKC activator is administered intravenously.

The present disclosure also relates to a method for activating a synaptic growth factor in a patient suffering from Rett syndrome comprising administering a pharmaceutically effective amount of a PKC activator to said patient, wherein the activation results in the prevention and/or reduction in neuronal death in said patient.

For example, the prevention and/or reduction in neuronal death in said patient results in a corrective and/or normalizing effect on the brain development in said patient suffering from Rett syndrome.

For example, the corrective and/or normalizing effect results in an abatement of symptoms arising from a muscular issue, a respiratory issue, a developmental issue, a behavioral issue, and/or a cognitive issue.

For example, the corrective and/or normalizing effect results in an abatement of symptoms arising from epilepsy, seizures, constipation, drooling, scoliosis, teeth grinding, and/or tremors.

For example, the synaptic growth factor is brain-derived neurotrophic factor (BDNF), insulin-like growth factor (IGF), and/or nerve growth factor (NGF).

For example, IGF is IGF-1.

For example, the PKC activator is bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, a bryolog, or any combination thereof.

For example, the PKC activator is bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, or any combination thereof.

For example, the PKC activator is bryostatin 1.

For example, the PKC activator further comprises one or more of a polyunsaturated fatty acid, a potassium channel activator, for example, diazoxide, a neristatin, for example, neristatin 1, or any other PKC activator described herein.

For example, the PKC activator is a polyunsaturated fatty acid.

For example, the PKC activator is a potassium channel activator.

For example, the PKC activator is a neristatin.

For example, the PKC activator is phorbol-12-myristate-13-acetate (PMA), okadaic acid, 1α,25-dihydroxyvitamin D3,12-deoxyphorbol-13-acetate (prostratin), 1,2-dioctanoyl-sn-glycerol (DOG), 1-oleoyl-2-acetyl-sn-glycerol (OAG), (2S,5S)-(E,E)-8-(5-(4-(trifluoromethyl)phenyl)-2,4-pentadienoylamino)benzolactam (α-amyloid precursor protein modulator), cis-9-octadecenoic acid (oleic acid), ingenol 3-angelate, resiniferatoxin, L-α-Phosphatidyl-D-myo-inosito1-4,5-bisphosphate, triammonium salt (PIP2), phorbol-12,13-dibutyrate, 8(S-hydroxy-(5Z, 9E,11Z,14Z)-eicosatetraenoic acid (8(S)-HETE), 12β-[(E,E)-5-Phenyl-2,4-pentadienoyloxy]daphnetoxin (merzerein), clomiphene citrate, sodium oleate, phorbol 12,13-diacetate, phorbol-12,13-didecanoate, 1,2-dipalmitoyl-sn-glycerol, 1-Stearoyl-2-linoleoyl-sn-glycerol, 1-stearoyl-2-linoleoyl-sn-glycerol, phorbol-12,13-dihexanoate, prostratin, a prostratin analog, resiniferonol 9,13,14-ortho-phenylacetate, C-8 ceramide, 1,6-bis(Cyclohexyloximinocarbonylamino)hexane; 1,6-Di(O-(carbamoyl)cyclohexanone oxime) hexane (RHC-80267), (+/−)-1-oleoyl-2-acetylglycerol, 5(S),6(R),15(S)-TriHETE (Lipoxin A4), (−)-Indolactam V, SC-9, SC-10, zoledronic acid monohydrate, 12-deoxyphorbo-13-angelate 20-acetate, 6-(N-decylamino)-4-hydroxymethylindole, 4α-phorbol 12,13-dibutyrate, 1,2-dihexanoyl-sn-glycerol, zoledronic acid disodium salt tetrahydrate, arachidonic acid methyl ester, or arachidonic acid-d8.

For example, the PKC activator activates the PKC ε isozyme and/or the PKC α isozyme.

For example, PKC activator activates the PKC ε isozyme.

For example, the PKC activator is administered orally, intraperitoneally, subcutaneously, intranasally, buccally, trans-dermally, intramuscularly, intrarectally, intravenously, or by inhalation.

For example, the PKC activator is administered orally.

For example, the PKC activator is administered intravenously.

In general, the present disclosure provides methods for treating Rett syndrome using PKC activators. As used herein, “protein kinase C activator” or “PKC activator” refers to a substance that increases the rate of the reaction catalyzed by protein kinase C, upregulates the expression of PKC (e.g., upregulates the expression of PKC α, PKC βII, PKC γ and/or PKC ε), or otherwise facilitates the activation of PKC.

In certain embodiments, the present disclosure provides methods comprising administering to a human subject with Rett syndrome a pharmaceutically effective amount of a PKC activator. The PKC activator may be administered as part of a composition suitable for administration to a human subject.

In certain embodiments, the PKC activator may be any of bryostatin 1-20, a bryolog, neristatin, a polyunsaturated fatty acid, or combinations thereof.

Bryostatins may be used in the methods of the present disclosure. The bryostatins are a family of naturally occurring macrocyclic compounds originally isolated from marine bryozoa. Currently, there are about 20 known natural bryostatins which share three six-membered rings designated A, B and C, and which differ mainly in the nature of their substituents at C7 (OR^{A}) and C20 (R^{B}). For example, in bryostatin 1, R^{A} is —C(═O)CH₃ (acetyl) and R^{B} is —OC(═O)CH═CH—CH═CH—C₃H₁₇. For example, in bryostatin 2, R^{A} is —H and R^{B} is —OC(═O)CH═CH—CH═CH—C₃H₇. For example, in bryostatin 4, R^{A} is —C(═O)-t-butyl and R^{B} is —OC(═O)n-propyl. For example, in bryostatin 5, R^{A} is —C(═O)-t-butyl and R^{B} is —OC(═O)CH₃. For example, in bryostatin 6, R^{A} is —C(═O)n-propyl and R^{B} is —OC(═O)CH₃ (acetyl). For example, in bryostatin 7, R^{A} is —C(═O)CH₃ and R^{B} is —OC(═O)CH₃. For example, in bryostatin 8, R^{A} is —C(═O)n-propyl and R^{B} is —OC(═O)n-propyl. For example, in bryostatin 9, R^{A} is —C(═O)CH₃ and R^{B} is —OC(═O)n-propyl.

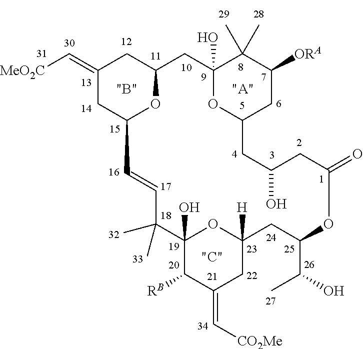

Bryostatin 1 and derivatives of bryostatin 1 are described in U.S. Pat. No. 4,560,774 (incorporated herein by reference). Examples of suitable bryostatins that may be used with the methods of the present disclosure include, bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, and bryostatin 20.

The terms “bryostatins” or “a bryostatin” are intended to include one or more of bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, and bryostatin 20.

“Bryologs,” i.e., analogs of bryostatins, may also be used in the methods of the present disclosure. Bryologs are structural analogues of bryostatin and have a reduced stability relative to bryostatin in both strong acid and base. However, at physiological pH, bryostatin and the bryologs exhibit similar stabilities. Bryologs also have a lower molecular weight (ranging from about 600 to 755), as compared to bryostatin (988), a property which may facilitate transport across the blood-brain barrier. Examples of suitable bryologs include, but are not limited to, analogs and derivatives of bryostatins such as those disclosed in U.S. Pat. Nos. 6,624,189, 7,256,286 and 8,497,385 (the disclosures of which are incorporated herein by reference).

In certain embodiments, polyunsaturated fatty acid esters (PUFAs or polyenoic fatty acids) may be used in the methods of the present disclosure for treating Rett syndrome. A PUFA is a fatty acid containing more than one double bond. There are three classes of PUFAs, omega-3 PUFAs, omega-6 PUFAs, and omega-9 PUFAS. In omega-3 PUFAs, the first double bond is found 3 carbons away from the last carbon in the chain (the omega carbon). In omega-6 PUFAs the first double bond is found 6 carbons away from the omega carbon and in omega-9 PUFAs the first double bond is 9 carbons from the omega carbon. As used herein, the term PUFA includes both naturally-occurring and synthetic fatty acids. A major source for PUFAs is from marine fish and vegetable oils derived from oil seed crops. Examples of PUFA's suitable for use in the methods of the present disclosure include, but are not limited to, esters of 8-[2-(2-pentylcyclopropylmethyl)cyclopropyl]-octanoic acid (DCPLA), as well as those described in U.S. Pat. No. 8,163,800 and in PCT Publication No. WO 2010/014585.

Another example of suitable PKC activators includes potassium channel activators such as, for example, diazoxide.

In certain embodiments, neristatins, such as neristatin 1, may be used in the methods of the present disclosure for treating a human subject with Rett syndrome.

Other suitable PKC activators include, but are not limited to, phorbol-12-myristate-13-acetate (PMA), okadaic acid, 1α,25-dihydroxyvitamin D3,12-deoxyphorbol-13-acetate (prostratin), 1,2-dioctanoyl-sn-glycerol (DOG), 1-oleoyl-2-acetyl-sn-glycerol (OAG), (2S,5S)-(E,E)-8-(5-(4-(trifluoromethyl)phenyl)-2,4-pentadienoylamino)benzolactam (α-amyloid precursor protein modulator), cis-9-octadecenoic acid (oleic acid), ingenol 3-angelate, resiniferatoxin, L-α-Phosphatidyl-D-myo-inositol-4,5-bisphosphate, triammonium salt (PIP2), phorbol-12,13-dibutyrate, 8(S-hydroxy-(5Z,9E,11Z,14Z)-eicosatetraenoic acid (8(S)-HETE), 12β-[(E,E)-5-Phenyl-2,4-pentadienoyloxy]daphnetoxin (merzerein), clomiphene citrate, sodium oleate, phorbol 12,13-diacetate, phorbol-12,13-didecanoate, 1,2-dipalmitoyl-sn-glycerol, 1-Stearoyl-2-linoleoyl-sn-glycerol, 1-stearoyl-2-linoleoyl-sn-glycerol, phorbol-12,13-dihexanoate, prostratin and its analogs, resiniferonol 9,13,14-ortho-phenylacetate, C-8 ceramide, 1,6-bis(Cyclohexyloximinocarbonylamino)hexane; 1,6-Di(O-(carbamoyl)cyclohexanone oxime) hexane (RHC-80267), (+/−)-1-oleoyl-2-acetylglycerol, 5(S),6(R),15(S)-TriHETE (Lipoxin A4), (−)-Indolactam V, SC-9, SC-10, zoledronic acid monohydrate, 12-deoxyphorbo-13-angelate 20-acetate, 6-(N-decylamino)-4-hydroxymethylindole, 4α-phorbol 12,13-dibutyrate, 1,2-dihexanoyl-sn-glycerol, zoledronic acid disodium salt tetrahydrate, arachidonic acid methyl ester, arachidonic acid-d8.

As used herein, “a pharmaceutically effective amount” is an amount of a pharmaceutical compound or composition having a therapeutically relevant effect on a human subject with Rett syndrome. For example, “a pharmaceutically effective amount” is an amount of a pharmaceutical compound or composition that activates one or more synaptic growth factors in a patient suffering from Rett syndrome, wherein the activation results in a corrective and/or normalizing effect on the brain development in said patient suffering from Rett syndrome.

As used herein, “corrective and/or normalizing effect” is a neutral or positive outcome in the brain development in a patient suffering from Rett syndrome. For example, this corrective and/or normalizing effect results in an abatement of symptoms, infra, and is the result of the administration of one or more PKC activators to the patient which activates a synaptic growth factor, e.g., BDNF, in a patient suffering from Rett syndrome.

The corrective and/or normalizing effect may relate to an abatement of symptoms arising from a muscular issue, for example: flaccid muscles, inability to combine muscle movements, muscle weakness, problems with coordination, stiff muscles, or rhythmic muscle contractions.

The corrective and/or normalizing effect may relate to an abatement of symptoms arising from a respiratory issue, for example: abnormal breathing patterns, episodes of no breathing, rapid breathing, or shallow breathing.

The corrective and/or normalizing effect may relate to an abatement of symptoms arising from a developmental issue, for example: delayed development or failure to thrive.

The corrective and/or normalizing effect may relate to an abatement of symptoms arising from a behavioral issue, for example: irritability or repetitive movements.

The corrective and/or normalizing effect may relate to an abatement of symptoms arising from a cognitive issue, for example: inability to speak or understand or slowness in activity and thought.

The corrective and/or normalizing effect may also relate to an abatement of symptoms arising from epilepsy, seizures, constipation, drooling, scoliosis, teeth grinding, and tremors.

In certain embodiments, a pharmaceutically effective amount for bryostatins and bryologs may be from about 0.0000001 to about 500 mg per kg host body weight per day, which can be administered in single or multiple doses. In some embodiments, the dosage level may be: from about 0.0000001 mg/kg to about 250 mg/kg per day, which can be administered in single or multiple doses; from about 0.0000005 mg/kg to about 100 mg/kg per day, which can be administered in single or multiple doses; from at least about 0.0000001 mg/kg to about 250 mg/kg per day, which can be administered in single or multiple doses; from at least about 0.00000005 mg/kg to about 100 mg/kg per day, which can be administered in single or multiple doses; from at least about 0.000001 mg/kg to about 50 mg/kg per day, which can be administered in single or multiple doses; or from about 0.00001 mg/kg to about 5.0 mg/kg per day, which can be administered in single or multiple doses. In other embodiments, the dosage may be about 0.00000001 mg/kg to about 0.00005 mg/kg per day, which can be administered in single or multiple doses; 0.00005 mg/kg to about 0.05 mg/kg per day, which can be administered in single or multiple doses; about 0.0005 mg/kg to about 5.0 mg/kg per day, which can be administered in single or multiple doses; about 0.0001 mg/kg to about 0.5 mg/kg per day, which can be administered in single or multiple doses; or 0.001 to 0.25 mg/kg per day, which can be administered in single or multiple doses.

In certain embodiments, a pharmaceutically effective amount for bryostatins and bryologs may be from about 0.0000001 to about 500 mg per kg host body weight per day, which can be administered IV in single or multiple doses. In some embodiments, the dosage level may be: from about 0.0000001 mg/kg to about 250 mg/kg per day, which can be administered IV in single or multiple doses; from about 0.0000005 mg/kg to about 100 mg/kg per day, which can be administered IV in single or multiple doses; from at least about 0.0000001 mg/kg to about 250 mg/kg per day, which can be administered IV in single or multiple doses; from at least about 0.00000005 mg/kg to about 100 mg/kg per day, which can be administered IV in single or multiple doses; from at least about 0.000001 mg/kg to about 50 mg/kg per day, which can be administered IV in single or multiple doses; or from about 0.00001 mg/kg to about 5.0 mg/kg per day, which can be administered in single or multiple doses. In other embodiments, the dosage may be about 0.00000001 mg/kg to about 0.00005 mg/kg per day, which can be administered IV in single or multiple doses; 0.00005 mg/kg to about 0.05 mg/kg per day, which can be administered IV in single or multiple doses; about 0.0005 mg/kg to about 5.0 mg/kg per day, which can be administered IV in single or multiple doses; about 0.0001 mg/kg to about 0.5 mg/kg per day, which can be administered IV in single or multiple doses; or 0.001 to 0.25 mg/kg per day, which can be administered in single or multiple doses.

In certain embodiments, bryostatin 1 may be from about 0.0000001 to about 500 mg per kg host body weight per day, which can be administered IV in single or multiple doses. In some embodiments, the dosage level may be: from about 0.0000001 mg/kg to about 250 mg/kg per day, which can be administered IV in single or multiple doses; from about 0.0000005 mg/kg to about 100 mg/kg per day, which can be administered IV in single or multiple doses; from at least about 0.0000001 mg/kg to about 250 mg/kg per day, which can be administered IV in single or multiple doses; from at least about 0.00000005 mg/kg to about 100 mg/kg per day, which can be administered IV in single or multiple doses; from at least about 0.000001 mg/kg to about 50 mg/kg per day, which can be administered IV in single or multiple doses; or from about 0.00001 mg/kg to about 5.0 mg/kg per day, which can be administered in single or multiple doses. In other embodiments, the dosage may be about 0.00000001 mg/kg to about 0.00005 mg/kg per day, which can be administered IV in single or multiple doses; 0.00005 mg/kg to about 0.05 mg/kg per day, which can be administered IV in single or multiple doses; about 0.0005 mg/kg to about 5.0 mg/kg per day, which can be administered IV in single or multiple doses; about 0.0001 mg/kg to about 0.5 mg/kg per day, which can be administered IV in single or multiple doses; or 0.001 to 0.25 mg/kg per day, which can be administered in single or multiple doses.

In certain embodiments, a pharmaceutically effective amount for bryostatins and bryologs may be from about 0.0000001 to about 500 mg per kg host body weight per day, which can be administered in single or multiple doses. In some embodiments, the dosage level may be: from about 0.0000001 mg/kg to about 250 mg/kg per day; from about 0.0000005 mg/kg to about 100 mg/kg per day; from at least about 0.0000001 mg/kg to about 250 mg/kg per day; from at least about 0.00000005 mg/kg to about 100 mg/kg per day; from at least about 0.000001 mg/kg to about 50 mg/kg per day; or from about 0.00001 mg/kg to about 5.0 mg/kg per dose. In other embodiments, the dosage may be about 0.00000001 mg/kg to about 0.00005 mg/kg; 0.00005 mg/kg to about 0.05 mg/kg; about 0.0005 mg/kg to about 5.0 mg/kg per day; about 0.0001 mg/kg to about 0.5 mg/kg per dose; or 0.001 to 0.25 mg/kg per dose.

In certain embodiments, the IV dosing is from about 1 μg/kg (3-25 μg/m²) to 120 μg/kg (360-3000 μg/m²). In other embodiments, the IV dosing is from about 0.04-0.3 μg/kg (1 μg/m²) to about 1-10 μg/kg (25 μg/m²). In other embodiments, the IV dosing is from about 0.01 μg/m² to about 25 μg/m². In other embodiments, the IV dosing is from about 0.0002-0.0004 μg/kg to about 0.05-1 μg/kg.

In certain embodiments, the PKC activator is a polyunsaturated fatty acid (PUFA) administered at a dosage of about 0.001 to 100 mg/kg, which can be administered in single or multiple doses; 0.01 to about 50 mg/kg, which can be administered in single or multiple doses; about 0.1 to about 10 mg/kg, which can be administered in single or multiple doses.

In certain embodiments, the PKC activator present in the compositions used in the methods of the present disclosure is a bryostatin, e.g., bryostatin 1, or bryolog, and the bryostatin or bryolog is used in an amount from about 0.0001 to about 1000 milligrams. In some embodiments, the bryostatin or bryolog is used in an amount from at least about 0.0001, 0.0005, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, or about 1000.0 milligrams.

In certain embodiments, the PKC activator present in the compositions used in the methods of the present disclosure is a bryostatin, e.g., bryostatin 1, or bryolog, and the bryostatin or bryolog is used in an amount from about 0.0001 to about 1000 milligrams. In some embodiments, the bryostatin or bryolog is used in an amount from about 0.0001, 0.0005, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, or about 1000.0 milligrams.

In certain embodiments, the PKC activator present in the compositions used in the methods of the present disclosure is a bryostatin, e.g., bryostatin 1, or bryolog, and the bryostatin or bryolog is used in an amount from about 0.0001 to about 1000 milligrams. In some embodiments, the bryostatin or bryolog is used in an amount of at least 0.0001, 0.0005, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, or about 1000.0 milligrams.

In certain embodiments, the PKC activator present in the compositions used in the methods of the present disclosure is a bryostatin, e.g., bryostatin 1, or bryolog, and the bryostatin or bryolog is used in an amount from about 0.0001 to about 1000 milligrams. In some embodiments, the bryostatin or bryolog is used in an amount of 0.0001, 0.0005, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1.0, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, or about 1000.0 milligrams.

The compositions used in the methods of the present disclosure may be administered via any suitable route; for example, orally, intraperitoneally, subcutaneously, intranasally, buccally, trans-dermally, intramuscularly, intrarectally, intravenously, and by inhalation. In one embodiment, the composition is administered intravenously. In one embodiment, the compositions is administered orally. In one embodiment, the compositions is administered intramuscularly.

The compositions used in the methods of the present disclosure may be administered on a regimen of 1 to 4 times per day, and in some embodiments, the compositions are administered twice a week, once a week, once every two weeks, once every three weeks, once every four weeks, once every six weeks, once every eight weeks or even less frequently depending on the needs of the patient.

The compositions used in the methods of the present disclosure may be administered as part of a course of treatment lasting for about 1 to about 30 days; about 1 to about 90 days; about 1 to about 120 days; about 1 to about 180 days; about 1 to 365 days; one year; two years; three years; or for the patient's lifetime.

It will be understood, however, that the specific dose level and frequency of dosage for any particular host may be varied and will depend upon a variety of factors including the activity of the specific compound employed, the metabolic stability and length of action of that compound, the age, body weight, general health, sex, diet, mode and time of administration, rate of excretion, drug combination, the nature of the disorder, the severity of the particular disorder, and the host undergoing therapy.

The present application also relates to the following:
A. A compound selected from bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, a bryolog, a polyunsaturated fatty acid, or a combination thereof, for use in the treatment of Rett syndrome.
B. The compound for use according to A, wherein the compound is selected from bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, or a combination thereof.
C. The compound for use according to A, wherein the compound is bryostatin 1.
D. The compound for use according to any one of A to C, wherein the compound or combination of compounds is used in a pharmaceutically effective amount.
E. The compound for according to D, wherein the pharmaceutically effective amount is from about 0.0000001 mg/kg to about 250 mg/kg per dose.
F. The compound for according to E wherein the pharmaceutically effective amount is from about 0.00001 mg/kg to about 5.0 mg/kg per dose.
G. The compound for use according to any one of D to F, wherein the pharmaceutically effective amount of bryostatin 1 is provided in a dose from 0.01-25 μg/m² intravenously.
H. A PKC activator for use in activating a synaptic growth factor in a patient suffering from Rett syndrome.
I. The PKC activator for use according to H, wherein the activation results in a corrective and/or normalizing effect on the brain development in said patient suffering from Rett syndrome.
J. The PKC activator for use according to H, wherein the activation results in an increase in the protein levels of synaptic growth factors in said patient.
K. The PKC activator for use according to J, wherein the increase in the protein levels of synaptic growth factors in said patient results in a corrective and/or normalizing effect on the brain development in said patient suffering from Rett syndrome
L. The PKC activator for use according to H, wherein the activation results in the prevention and/or reduction in neuronal death in said patient.
M. The PKC activator for use according to L, wherein the prevention and/or reduction in neuronal death in said patient results in a corrective and/or normalizing effect on the brain development in said patient suffering from Rett syndrome.
N. The PKC activator for use according to I, K or M, wherein the corrective and/or normalizing effect results in an abatement of symptoms arising from a muscular issue, a respiratory issue, a developmental issue, a behavioral issue, and/or a cognitive issue.
O. The PKC activator for use according to I, K, M or N, wherein the corrective and/or normalizing effect results in an abatement of symptoms arising from epilepsy, seizures, constipation, drooling, scoliosis, teeth grinding, and/or tremors.
P. The PKC activator for use according to any one of H to O, wherein the PKC activator is administered in a pharmaceutically effective amount.
Q. The PKC activator for use according to any one of H to P, wherein the synaptic growth factor is brain-derived neurotrophic factor (BDNF), insulin-like growth factor (IGF), and/or nerve growth factor (NGF).
R. The PKC activator for use according to, Q wherein the IGF is IGF-1.
S. The PKC activator for use according to any one of H to R, wherein the PKC activator is bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, a bryolog, or any combination thereof.
T. The PKC activator for use according to S, wherein the PKC activator is bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, or any combination thereof.
U. The PKC activator for use according to T, wherein the PKC activator is bryostatin 1.
V. The PKC activator for use according to any one of S to U, wherein the PKC activator further comprises one or more of a polyunsaturated fatty acid, a potassium channel activator, for example, diazoxide, a neristatin, for example, neristatin 1, or any other PKC activator described herein.
W. The PKC activator for use according to any one of H to R, wherein the PKC activator is a polyunsaturated fatty acid.
X. The PKC activator for use according to any one of H to R, wherein the PKC activator is a potassium channel activator.
Y. The PKC activator for use according to any one of H to R, wherein the PKC activator is a neristatin.
Z. The PKC activator for use according to any one of H to R, wherein the PKC activator is phorbol-12-myristate-13-acetate (PMA), okadaic acid, 1α,25-dihydroxyvitamin D3,12-deoxyphorbol-13-acetate (prostratin), 1,2-dioctanoyl-sn-glycerol (DOG), 1-oleoyl-2-acetyl-sn-glycerol (OAG), (2S,5S)-(E,E)-8-(5-(4-(trifluoromethyl)phenyl)-2,4-pentadienoylamino) benzolactam (α-amyloid precursor protein modulator), cis-9-octadecenoic acid (oleic acid), ingenol 3-angelate, resiniferatoxin, L-α-Phosphatidyl-D-myo-inositol-4,5-bisphosphate, triammonium salt (PIP2), phorbol-12,13-dibutyrate, 8(S-hydroxy-(5Z,9E,11Z,14Z)-eicosatetraenoic acid (8(S)-HETE), 12β-[(E,E)-5-Phenyl-2,4-pentadienoyloxy]daphnetoxin (merzerein), clomiphene citrate, sodium oleate, phorbol 12,13-diacetate, phorbol-12,13-didecanoate, 1,2-dipalmitoyl-sn-glycerol, 1-Stearoyl-2-linoleoyl-sn-glycerol, 1-stearoyl-2-linoleoyl-sn-glycerol, phorbol-12,13-dihexanoate, prostratin, a prostratin analog, resiniferonol 9,13,14-ortho-phenylacetate, C-8 ceramide, 1,6-bis(Cyclohexyloximinocarbonylamino)hexane; 1,6-Di(O-(carbamoyl)cyclohexanone oxime) hexane (RHC-80267), (+/−)-1-oleoyl-2-acetylglycerol, 5(S),6(R),15(S)-TriHETE (Lipoxin A4), (−)-Indolactam V, SC-9, SC-10, zoledronic acid monohydrate, 12-deoxyphorbo-13-angelate 20-acetate, 6-(N-decylamino)-4-hydroxymethylindole, 4α-phorbol 12,13-dibutyrate, 1,2-dihexanoyl-sn-glycerol, zoledronic acid disodium salt tetrahydrate, arachidonic acid methyl ester, or arachidonic acid-d8.
AA. The PKC activator for use according to any one of H to Z, wherein the PKC activator activates the PKC ε isozyme and/or the PKC α isozyme.
BB. The PKC activator for use according to any one of H to AA, wherein the PKC activator activates the PKC ε isozyme.
CC. The PKC activator for use according to any one of H to BB, wherein the PKC activator is administered orally, intraperitoneally, subcutaneously, intranasally, buccally, trans-dermally, intramuscularly, intrarectally, intravenously, or by inhalation.
DD. The PKC activator for use according to any one of H to CC, wherein the PKC activator is administered orally.
EE. The PKC activator for use according to any one of H to CC, wherein the PKC activator is administered intravenously.

Also, various inventive concepts may be embodied as one or more methods or pharmaceutical compositions for use, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All publications, patent applications, patents, and other references mentioned herein are incorporated by reference in their entirety.

Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

All definitions, as defined and used herein, should be understood to control over dictionary definitions, definitions in documents incorporated by reference, and/or ordinary meanings of the defined terms.

The compositions disclose herein, may contain one or more pharmaceutically acceptable excipient, which comprises any of the following classes of ingredients: fillers, binders, lubricants, disintegrating agents, glidants (e.g., silicon dioxide), flavoring agents and colorants. Suitable binders include, e.g., microcrystalline cellulose (e.g., Avicel PH200 LM, PH112, PH101, PH102, PH103, PH113, PH105, PH200, DG), mannitol, dicalcium phosphate, dicalcium phosphate anhydrous, povidone, lactose, glucose, starch, gelatin, acacia gum, tragacanth gum, sodium alginate, carboxymethylcellulose, polyethylene glycol, waxes or the like. Lubricants include, e.g., glyceryl dibehenate, hydrogenated vegetable oil, sodium oleate, sodium stearate, magnesium stearate, silicon dioxide, sodium benzoate, sodium acetate, sodium chloride or the like. Other excipients include, e.g., starch, methyl cellulose, agar, bentonite, xanthan gum, sodium starch glycolate, crospovidone, croscarmellose sodium or the like. Additional excipients for capsules include macrogols or lipids and/or any other excipients known in the art. These examples are not intended to be limiting.

Any of the compositions or pharmaceutical compositions described herein may be formulated with pharmaceutically acceptable carriers or diluents as well as any other known adjuvants and excipients in accordance with conventional techniques such as those disclosed in Remington: The Science and Practice of Pharmacy, 21^{st} Edition, 2000, Lippincott Williams & Wilkins, which is incorporated herein in its entirety.

The term “about,” as used herein, and unless explicitly stated otherwise, refers to a recited value +/−10%, +/−5%, +/−2.5%, +/−1%, or +/−0.5%. For example, “about” may refer to a recited value +/−5%.

The term, “subject” as used herein refers to a human or non-human, i.e., a patient. In one embodiment, the subject is a mammal. In one embodiment, the subject is a human.

The phrase, “therapeutically effective amount” or “effective amount” as used herein indicates an amount necessary to administer to a subject, or to a cell, tissue, or organ of a subject, to achieve a therapeutic effect, such as an ameliorating or alternatively a curative effect.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein, phrases containing the term “and/or” such as “A, B and/or C” refer to any of the following: A only; B only; C only; A and B; A and C; B and C; A, B and C.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1.-6. (canceled)

7. A method for activating a synaptic growth factor in a patient suffering from Rett syndrome comprising administering a pharmaceutically effective amount of a PKC activator to said patient, wherein the activation results in a corrective and/or normalizing effect on the brain development in said patient suffering from Rett syndrome.

8. The method of claim 7, wherein the synaptic growth factor is brain-derived neurotrophic factor (BDNF), insulin-like growth factor (IGF), and/or nerve growth factor (NGF).

9. The method claim 8, wherein the IGF is IGF-1.

10. (canceled)

11. The method of claim 7, wherein the PKC activator is bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, a bryolog, or any combination thereof.

12. (canceled)

13. The method of claim 11, wherein the PKC activator further comprises a polyunsaturated fatty acid, a potassium channel activator, a neristatin, or any combination thereof.

14. The method of claim 7, wherein the PKC activator is a polyunsaturated fatty acid, a potassium channel activator, or a neristatin.

15.-16. (canceled)

17. The method of claim 7, wherein the PKC activator is phorbol-12-myristate-13-acetate (PMA), okadaic acid, 1α,25-dihydroxyvitamin D3, 12-deoxyphorbol-13-acetate (prostratin), 1,2-dioctanoyl-sn-glycerol (DOG), 1-oleoyl-2-acetyl-sn-glycerol (OAG), (2S,5S)-(E,E)-8-(5-(4-(trifluoromethyl)phenyl)-2,4-pentadienoylamino) benzolactam (α-amyloid precursor protein modulator), cis-9-octadecenoic acid (oleic acid), ingenol 3-angelate, resiniferatoxin, L-α-Phosphatidyl-D-myo-inositol-4,5-bisphosphate, triammonium salt (PIP2), phorbol-12,13-dibutyrate, 8(S-hydroxy-(5Z,9E,11Z,14Z)-eicosatetraenoic acid (8(S)-HETE), 12β-[(E,E)-5-Phenyl-2,4-pentadienoyloxy]daphnetoxin (merzerein), clomiphene citrate, sodium oleate, phorbol 12,13-diacetate, phorbol-12,13-didecanoate, 1,2-dipalmitoyl-sn-glycerol, 1-Stearoyl-2-linoleoyl-sn-glycerol, 1-stearoyl-2-linoleoyl-sn-glycerol, phorbol-12,13-dihexanoate, prostratin, a prostratin analog, resiniferonol 9,13,14-ortho-phenylacetate, C-8 ceramide, 1,6-bis(Cyclohexyloximinocarbonylamino)hexane; 1,6-Di(O-(carbamoyl)cyclohexanone oxime) hexane (RHC-80267), (+/−)-1-oleoyl-2-acetylglycerol, 5(S),6(R),15(S)-TriHETE (Lipoxin A4), (−)-Indolactam V, SC-9, SC-10, zoledronic acid monohydrate, 12-deoxyphorbo-13-angelate 20-acetate, 6-(N-decylamino)-4-hydroxymethylindole, 4α-phorbol 12,13-dibutyrate, 1,2-dihexanoyl-sn-glycerol, zoledronic acid disodium salt tetrahydrate, arachidonic acid methyl ester, or arachidonic acid-d8.

18. The method of claim 7, wherein the PKC activator activates the PKC ε isozyme and/or the PKC α isozyme.

19. (canceled)

20. The method of claim 7, wherein the PKC activator is administered orally, intraperitoneally, subcutaneously, intranasally, buccally, transdermally, intramuscularly, intrarectally, intravenously, or by inhalation.

21.-22. (canceled)

23. The method of claim 7, wherein the corrective and/or normalizing effect results in an abatement of symptoms arising from a muscular issue, a respiratory issue, a developmental issue, a behavioral issue, and/or a cognitive issue.

24. The method of claim 7, wherein the corrective and/or normalizing effect results in an abatement of symptoms arising from epilepsy, seizures, constipation, drooling, scoliosis, teeth grinding, and/or tremors.

25. A method for activating a synaptic growth factor in a patient suffering from Rett syndrome comprising administering a pharmaceutically effective amount of a PKC activator to said patient, wherein the activation results in an increase in the protein levels of synaptic growth factors in said patient.

26. The method of claim 25, wherein the increase in the protein levels of synaptic growth factors in said patient results in a corrective and/or normalizing effect on the brain development in said patient suffering from Rett syndrome.

27. The method of claim 26, wherein the corrective and/or normalizing effect results in an abatement of symptoms arising from a muscular issue, a respiratory issue, a developmental issue, a behavioral issue, and/or a cognitive issue.

28. The method of claim 26, wherein the corrective and/or normalizing effect results in an abatement of symptoms arising from epilepsy, seizures, constipation, drooling, scoliosis, teeth grinding, and/or tremors.

29. A method for activating a synaptic growth factor in a patient suffering from Rett syndrome comprising administering a pharmaceutically effective amount of a PKC activator to said patient, wherein the activation results in the prevention and/or reduction in neuronal death in said patient.

30. The method of claim 29, wherein the prevention and/or reduction in neuronal death in said patient results in a corrective and/or normalizing effect on the brain development in said patient suffering from Rett syndrome.

31. The method of claim 30, wherein the corrective and/or normalizing effect results in an abatement of symptoms arising from a muscular issue, a respiratory issue, a developmental issue, a behavioral issue, and/or a cognitive issue.

32. The method of claim 30, wherein the corrective and/or normalizing effect results in an abatement of symptoms arising from epilepsy, seizures, constipation, drooling, scoliosis, teeth grinding, and/or tremors.

33.-34. (canceled)

35. The method of claim 25, wherein the PKC activator is bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, a bryolog, or any combination thereof.

36. The method of claim 29, wherein the PKC activator is bryostatin 1, bryostatin 2, bryostatin 3, bryostatin 4, bryostatin 5, bryostatin 6, bryostatin 7, bryostatin 8, bryostatin 9, bryostatin 10, bryostatin 11, bryostatin 12, bryostatin 13, bryostatin 14, bryostatin 15, bryostatin 16, bryostatin 17, bryostatin 18, bryostatin 19, bryostatin 20, a bryolog, or any combination thereof.

37.-42. (canceled)

43. The method of claim 25, wherein the PKC activator activates the PKC ε isozyme and/or the PKC α isozyme.

44. The method of claim 29, wherein the PKC activator activates the PKC ε isozyme and/or the PKC α isozyme.

45.-50. (canceled)
